(19) United States
(12) Patent Application Publication
(10) Pub. No.: US 2009/0112321 A1
(43) Pub. Date: Apr. 30, 2009

(54) Spinal stabilization device and method

(76) Inventor: Michael S. Kitchen, Charleston, SC (US)

Correspondence Address: B. CRAIG KILLOUGH, P. O. DRAWER H, CHARLESTON, SC, 29402, US

(21) Appl. No.: 12/288,272

(22) Filed: Oct. 17, 2008

Related U.S. Application Data
(63) Continuation-in-part of application No. 11/196,891, filed on Aug. 4, 2005.
(60) Provisional application No. 60/980,667, filed on Oct. 17, 2007.

(51) Int. Cl. A61F 2/44; A61B 17/70

(52) U.S. Cl. 623/17.11; 606/246

(57) ABSTRACT

A thermally active member comprises a shape memory material. The method of placement of the thermally active member facilitates transition of shape memory material between a simple undefined geometry, such as a substantially linear shape, to a complex predetermined shape memory form, such as a coiled shape. The preferred complex geometry may be described as an intervertebral cage or cage fusion device. The device may be placed into a space surgically created between two vertebrae through an access channel that is less than one third of its final deployed cross sectional dimension.

### PRIORITY CLAIM

This application is a continuation in part of application Ser. No. 11/196,891 filed Aug. 4, 2005.

Applicant claims the benefit of provisional application Ser. No. 60/980,667 filed Oct. 17, 2007.

### FIELD OF THE INVENTION

The device and method relates to instrumentation for the mammalian spine and is particularly directed to a device for correction of degenerative, congenital, or traumatic deformity, and a method of placement of the device.

### BACKGROUND OF THE INVENTION

There is a need for a cage system capable of effecting rigid intervertebral fusion placed in a minimally invasive manner through a posterior-lateral approach without requiring laminectomy to effect placement.

There is a need for a cage system that may be placed in a minimally invasive manner that is capable of effecting dynamic stabilization to intervertebral segments.

There is a need for a cage fusion or dynamic stabilization device that may be placed into an intervertebral osteotomy through a posterior-lateral approach without utilization of a rigid tube access device.

There is a need for a cage fusion type device that may be utilized in the cervical region through an anterior-lateral approach with a minimally invasive technique for placement that does not require the use of rigid tube placement instrumentation.

There is a need for a cage fusion or dynamic stabilization device that may be placed at all spinal levels utilizing a minimally invasive technique for placement having small access requirements and instrumentation that is flexible in nature allowing for adaptation to anatomic and placement route variation.

There is a need for a cage type fusion/stabilization device that can be placed between vertebral bodies with minimal disruption of endogenous structures, thus maximizing retention of endogenous structure and stability, with little or no disruption of the annular ligament.

There is a need for a minimally invasive cage type device that may be placed utilizing a true lateral access technique with the cage device placed transversely across the vertebral bodies and without material disruption of muscular structures and without associated morbidity at the time of surgery.

There is a need for a device that can effect dynamic stabilization between vertebral segments.

There is a need for devices effecting dynamic stabilization between vertebral segments having adaptive structural capabilities that permit dynamic movement of a load axis mimicking the structural capabilities of a native intervertebral disc.

There is a need for a dynamic stabilization device that has a capability of avoiding failure through design of redundant load carrying members.

### SUMMARY OF THE INVENTION

A thermally active member comprises a shape memory material. The method of placement of the thermally active member facilitates transition of shape memory material between a simple undefined geometry, such as a substantially linear shape, to a complex predetermined shape memory form, such as a coiled shape. The preferred complex geometry may be described as an intervertebral cage or cage fusion device. The device may be placed into a space surgically created between two vertebrae through an access channel that is less than one third of its final deployed cross sectional dimension.

### DESCRIPTION OF THE DRAWINGS OF PREFERRED EMBODIMENTS

FIG. 1 demonstrates a device within a deployment catheter, with a pre-deployment portion shown in essentially linear form, and a post-deployment portion shown as transitioned to coiled form.

FIGS. 2 A, 2 B, 2C, 2 D demonstrate potential post-deployment shapes of the device including a level cylindrical form, a tapered conical form, and a lozenge shaped form.

FIG. 3 demonstrates an embodiment comprising triple helix geometry, wherein three parallel helices are fused at their ends maintaining register between the three components through the deployment process.

FIG. 3 B demonstrates a triple helix design undergoing deployment wherein the axis of the deploying device is parallel to the central axis of the deployment catheter

FIG. 4 demonstrates a cross-section of the device within a deployment catheter, and showing the heat transition zone of the deployment catheter.

FIG. 5 demonstrates the device fully deployed within a corresponding shaped osteotomy.

FIG. 6 demonstrate the contrast in form between the device as a simple linear geometry at lower temperature (FIG. 6A) and the higher temperature “shape memory” complex geometry of the fully deployed device (FIG. 6B).

FIG. 7 shows a cross section configuration showing a triple helix design having interdigitating wires, intended to minimize cross section size pre-deployment.

FIG. 8 demonstrates an essentially cylindrical geometry of the device with compressible side wall segments allowing for slight compression of the device and dynamic motion between adjacent vertebral bodies. Those portions of the device oriented towards the superior and inferior end plates of the vertebral bodies are covered with a polymer section designed to enhance fusion probability and/or to sequester wear debris.

FIG. 9 shows a sidewall of attenuated structure in a cage embodiment, with micro-formed slots in a helical component. The slots allow greater flexion of the structure for a given load in compression.

FIG. 10 shows an embodiment where the final shape memory form of the device is laminated, having two essentially independent devices placed sequentially one inside the other with a common central axis. This arrangement permits utilization of a relatively light structural device that gains structural strength through redundancy.

FIG. 11 shows the device having an outer surface for promotion of boney fusion to the inferior and superior vertebral bodies between which the device is deployed.

FIG. 12 shows the helical element(s) of the device with a polymer coating. The coating may serve to inhibit creation of wear debris or serve to sequester accumulated wear debris thus precluding the opportunity for immune reaction to occur.

FIG. 13 The device is shown deployed within a containment component which consists of a substantially impermeable membrane into which the device has been deployed. The impermeable membrane conforms to the shape of the surgically created osteotomy.

### DESCRIPTION OF PREFERRED EMBODIMENTS

The device of the preferred embodiments may be classified as a “cage fusion” type device. The device may comprise one or more structural components comprised of a shape memory material.

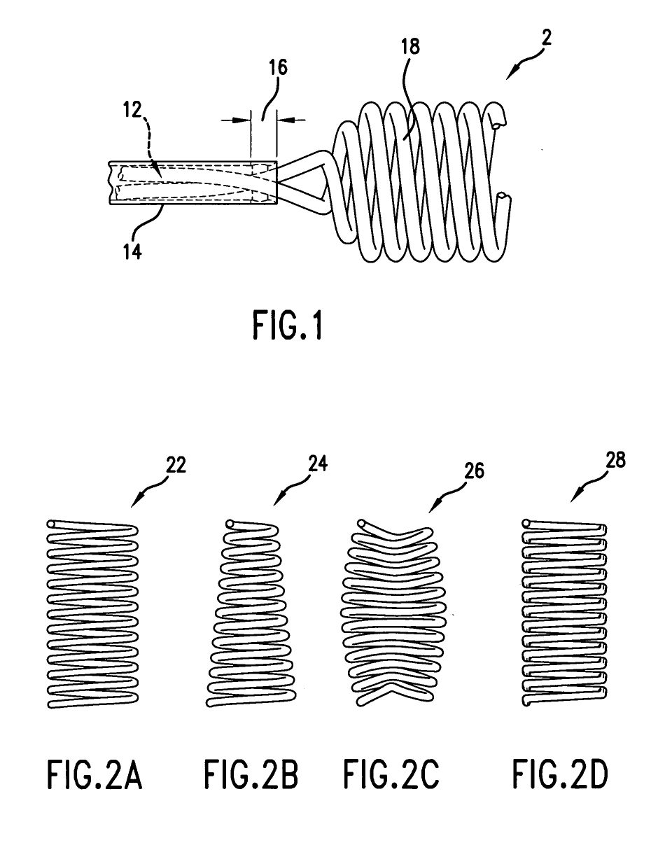

In pre-deployment form, the device components exist as simple elements that are malleable and capable of adopting an essentially linear form 12. FIG. 1. This linear form, which may be similar to a length of malleable wire, is capable of being moved through a lumen of a catheter 14, trocar, arthroscope, or similar deployment device in a linear progression. The deployment device may utilize the “Thermal Method” of deployment described in U.S. Patent Application Publication No. 20060030933 to effect an orderly, controlled and sequential transition between two states of a shape memory material. This controlled and sequential transition is preferred to occur by the application of heat to the device in a narrowly defined space 16.

The transitioned final form FIG. 1 of the device 2 is realized in situ and is capable of adopting a myriad of complex three dimensional forms. The device as positioned for use in the spine is preferred to be a coiled structure, and may be helical in structure. Exemplary coiled structures are shown in FIG. 2. The device may be formed as a level cylinder 22 (FIG. 2 A), a conic cylinder 24 (FIG. 2 B), a lozenge shape 26 (FIG. 2 C), and configured with a rectangular cross section 28 (FIG. 2 D). As long as the final form is maintained at temperatures above the design transition temperature, the device will remain in its super-elastic austenite “shape memory” or determinate form with high strength structural capabilities.

The device is constructed to maintain a super-elastic form at body temperature, and may assume this shape at slightly below body temperature. This final deployed form of the device has a shape and size that may be a cage type structure, with the cage providing a framework for support. The structural properties of the device, when maintained at temperatures at or above the transition temperature, which is preferred to be at, or slightly below, body temperature is able to correct or assist in correcting anatomic deformity between vertebral segments. The device can effect a rigid fusion between adjacent vertebral bodies, and is preferred to have a high degree of rigidity.

A further embodiment of the device provides structural scaling of material and cross-sections, and allows for design of a device with a predictable degree of compressibility, and creation of a dynamic stabilization construct. This embodiment may function as a prosthetic disc accommodating a controlled degree of motion between vertebral bodies. The geometry of this embodiment is capable of motion in six planes, and can emulate mechanical characteristics of native disc structures.

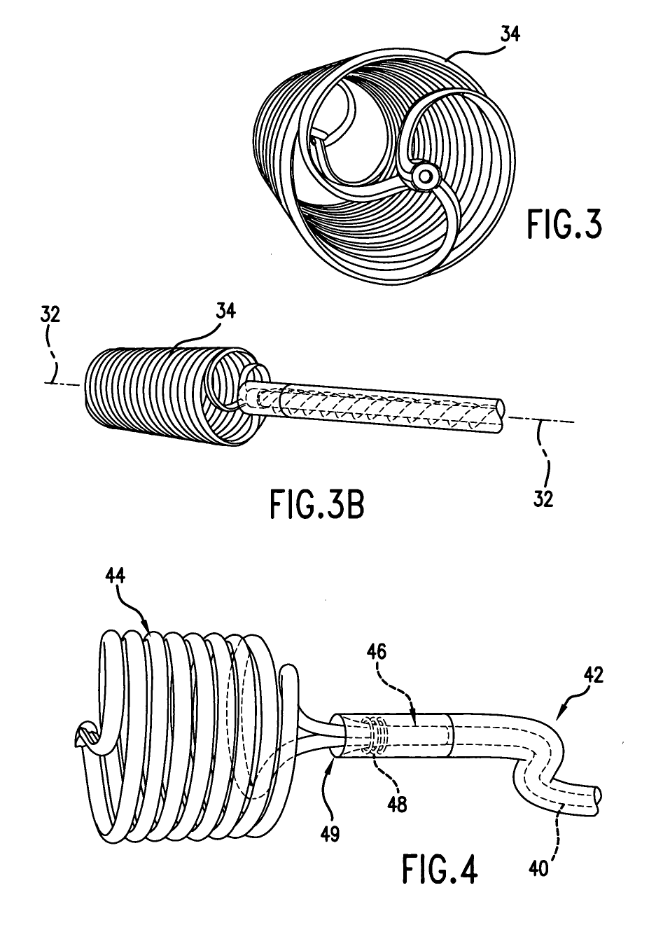

Parallel coiled or helical designs may be utilized. Two or more equivalent coils or helices possess the geometric property of deployment generally parallel to the central axis of a deployment means, such as a deployment lumen or catheter. The multiple parallel helices may be joined at proximal and/or distal ends, structurally maintaining register between the helices through the deployment process. A single helix structure may deploy tangentially across the central axis of the deployment catheter, and not yield a symmetric deployment process. A three parallel helix embodiment 34 allows for a fully structurally symmetric process of deployment, wherein the forces of transition between the three elements are radially balanced, yielding a geometric relationship that tends towards deployment parallel 32 to the catheters central axis. FIGS. 3 and 3B.

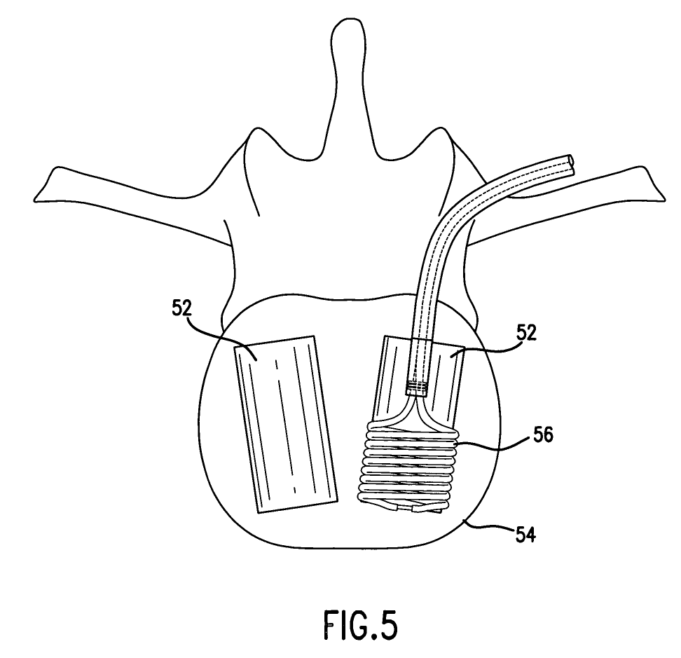

A thermal element comprises a thermally active shape memory material having super-elastic properties at body temperature. The design transition temperature may be specified below body temperature, such as 2 to 3 degrees Celsius below body temperature of the vertebrate into which the device is placed. Currently available materials meeting desirable specifications are various alloys of nitinol or nitinol like alloys. Alloy composition may be adjusted, creating shape memory materials having super-elastic and shape set characteristics (austenite state) near body temperature, while retaining those shape characteristics at body temperature and higher temperatures. These alloys exist at lower temperatures in martensite state wherein the material is relatively malleable and has no shape set or super-elastic properties, the shape may be expressed as “indeterminate” at these temperatures. When the shape of the device is indeterminate, if a dynamic force is placed upon the device and the dynamic force changes the shape, the shape into which the device is changed is retained when the dynamic force is removed. This martensite state corresponds to the pre-deployment malleable form, or indeterminate form, of the device. In this state, the device may be linear, like a wire, and may be bent 42 or shaped like a wire. In a preferred embodiment, the wire may be shaped manually by a physician installing the device. FIG. 4.

When heat is applied to the device, the device assumes its predetermined super-elastic austenite “shape memory” form with high strength structural capabilities 44. The device will retain this shape as long as the temperature is maintained above the predetermined temperature, which is preferred to be just below body temperature of the human or other vertebrate into which the device is to be positioned. When a dynamic force is not being actively applied to the device at this higher temperature, the device assumes and retains a predetermined shape, which may be summarily referred to as a determinate shape. The device is shown in various embodiments of determinate shape in the drawing Figures.

In one embodiment, the device is a wire 40 having a substantially round cross section. The determinate form of the device is shape set to a coiled or helical form. FIG. 4. The wire is maintained at a temperature below M_{f} (martensite final state) within the deployment catheter prior to placement 42. (At this temperature, the wire is readily formable with little force required to bend or shape the wire, and the wire may be pushed through a lumen of a flexible tube 46. The tip 49 of a catheter may be introduced to the depth of the cylindrical osteotomy. Heat is then introduced at the catheter tip, such as by the use of electrical resistance coils 48, transitioning the shape of the memory material to its determinate shape 44 as it is exposed to heat and as it exits the tip of the catheter. The temperature environment proximal to the catheter tip is maintained below M_{f}. Temperatures after the catheter tip are maintained at greater than A_{f} (body temperature or slightly below). Transition to austenite form proceeds linearly along the length of the device in an antigrade fashion: distal to proximal.

The device may be repositioned during the placement process by terminating heat introduction, and pulling the device in the opposite direction and into the catheter, where the temperature environment is less than M_{f}. Stated otherwise, the transition process is reversed.

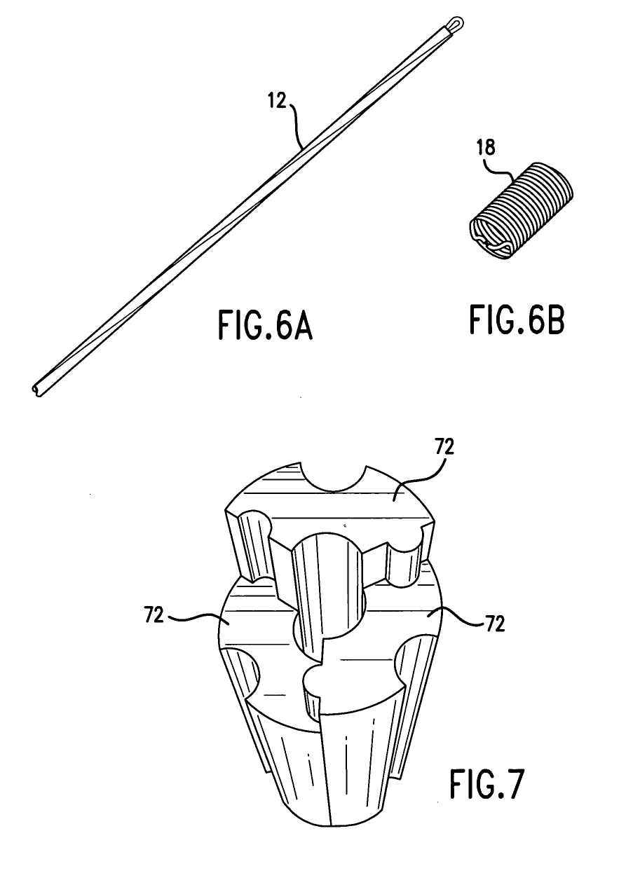

The final shape set form of the device is designed to substantially match the geometry of the osteotomy 52 formed for its placement in a vertebral body 54 (or usually, two vertebral bodies. FIG. 5. As transition occurs to austenite form, the device occupies the void 56 of the osteotomy in a distal to proximal fashion.

Geometric configurations of the device are not limited to simple cylindrical shapes. A specified final design shape may be reduced to a single or multiple linear components, and the device is amenable to placement utilizing the technique described herein. At a low temperature state the device has no intrinsic shape beyond its cross section and no super-elastic properties. The device at the low temperature state may be characterized as shape indeterminate or indefinite. In this temperature state, the shape of the device is indeterminate and may be formed into a linear shape having a cross section that is considerably smaller than the cross section of the deployed device. FIG. 6 A. It is preferred that the device is manually deformable at will to accommodate deployment. In sharp contrast to the low temperature state, in the higher temperature state the device has a specific size, shape, and super-elastic properties. This state of the device at the higher temperature is the “shape memory” or determinate form of the device. FIG. 6 B. As the device transitions between these two different temperature states, virtually any shape set final austenite form is attainable. Examples are shown in FIG. 2 C, 2 D, 2 E, 2 F, but these embodiments are by no means exhaustive of the possibilities.

This process permits low temperature martensite states to be utilized having non-complex geometry, and simple linear or substantially linear shapes, like a wire. The cross-section shape of these elements may be of any imaginable design, and especially those which may be extruded. The structural cross-section may likewise be varied along the length of a component to meet varied structural requirements for different portions of the component in its final “shape memory” or determinate form. Cross section variation allows not only for the device to meet structural requirements in its final deployed “shape memory” or determinate form, but permits design of the individual helical elements in interdigitating configurations 72, preferably yielding a small cross section of the device in its low temperature pre-deployment or indeterminate form. FIG. 7.

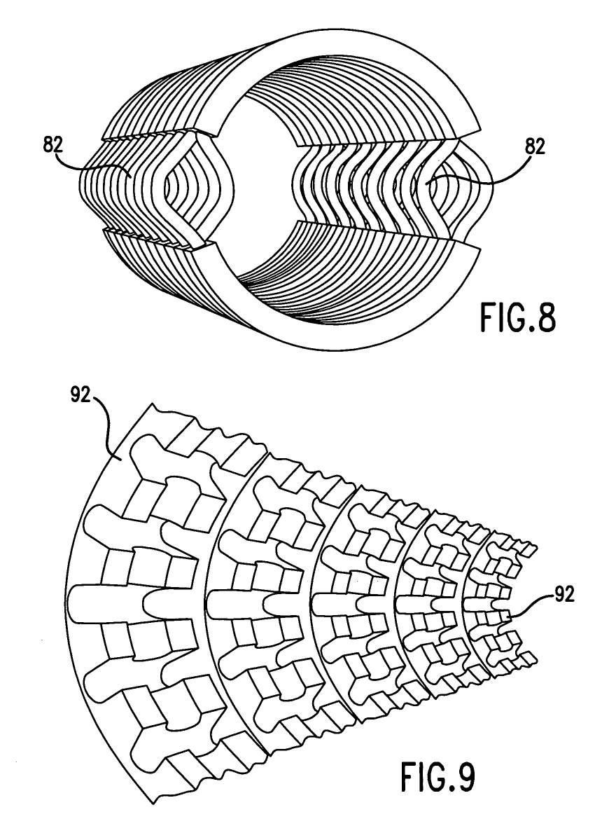

The design of the device may be specifically tailored to meet structural requirements for dynamic stabilization. Portions of the device may be so designed to allow compression between vertebral bodies and a limited degree of rotation between vertebral bodies; an essentially cylindrical geometry device with bent side wall segments 82 is shown which accommodate limited motion between vertebral segments. FIG. 8. In this embodiment, the cross section of the individual members may remain consistent throughout the device. The selective bending at specified areas creates a condition of structural attenuation.

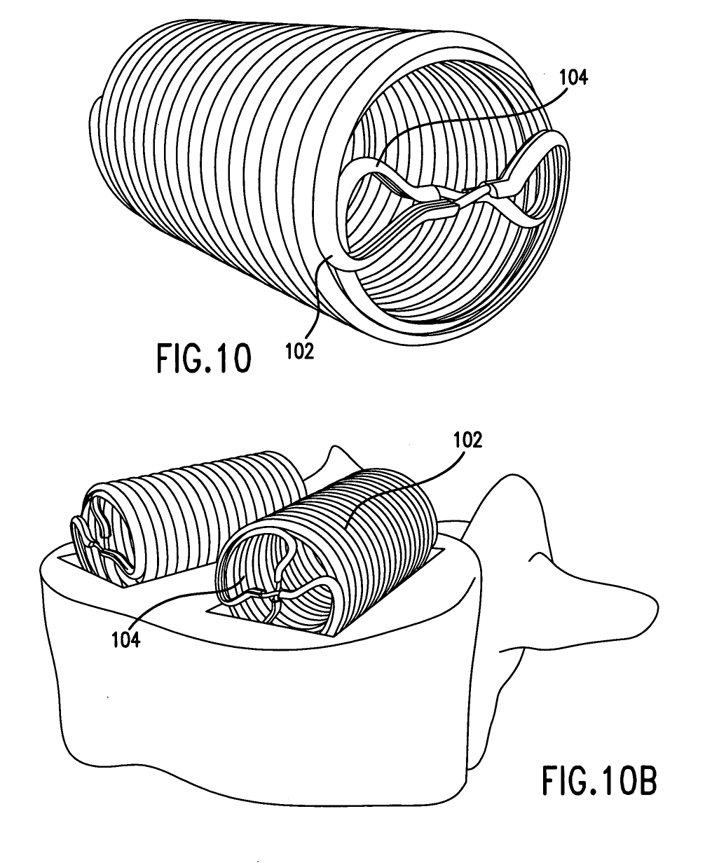

Further, the device may be so designed that specified portions of the structure are attenuated to allow a controlled degree of deformation to occur. FIG. 9. This embodiment may utilize micro machining techniques to remove portions of the structure 92, resulting in the formation of areas that undergo intended deformation in response to less force than the overall structure. These areas may be subject to relative ease of deformation in bending, twisting, compression or elongation when compared to the “normal” structural portions of the device. The device may be configured such that there is a plurality of independent cylindrical components arranged in a concentric configuration. FIG. 10. This embodiment involves sequential placement of two or more “devices” along a common central axis. One device is placed in the usual manner 102; a second is then placed inside the first 104 yielding an overall construct with structural properties greater than any of its individual components. In this embodiment, each successive cylindrical component contributes greater strength to the overall construct. Similar to the previously described embodiments this aspect of design may be adapted to multiple shapes, including a level cylinder, tapered conic form, or lozenge shape. This embodiment of the device as a laminated structure with each independent component placed sequentially permits use of a lighter structure for each of the separate cylindrical components, thereby permitting greater ease of deployment. Further, this design increases redundancy of structural elements allowing for smaller cross sections for each of the components and less consequence in the event of failure of any one or more structural elements. This embodiment as shown yields twice as many structural elements, since each complete circular element of the coil 102,104 is placed as a two layered system, as compared to a single layered system, such as that shown in FIG. 2.

The surfaces of the fully deployed device may be machined to produce a textured surface 112 that may increase the probability of boney fusion occurring. FIG. 11. This aspect of design may also be so configured that the individual coiled or helical elements have attenuated structural properties, allowing greater ease of bending, and facilitating passage of the low temperature shape indeterminate form of the device through the deployment catheter.

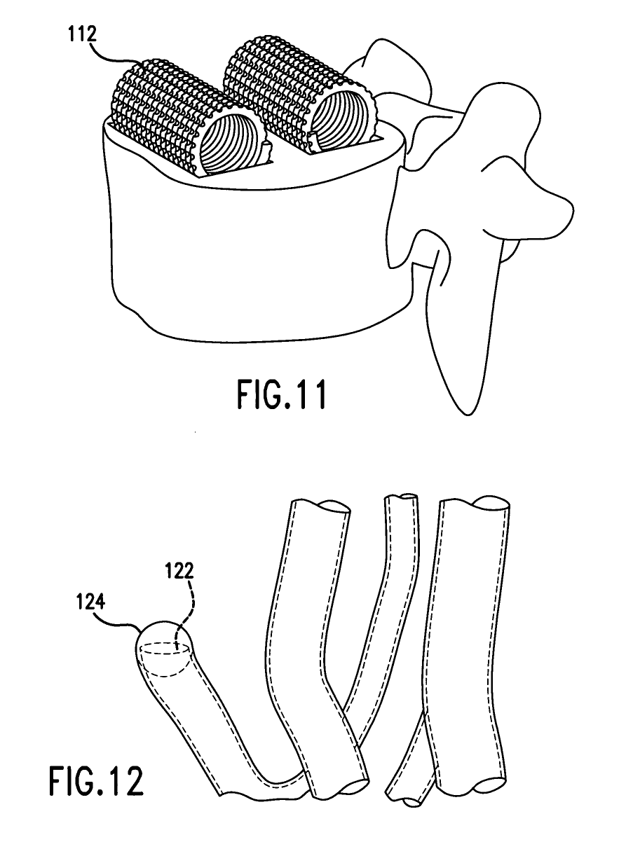

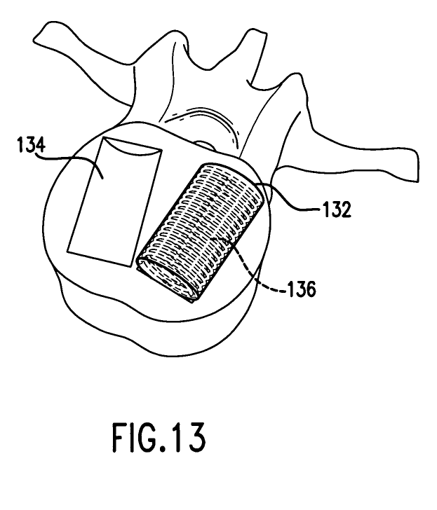

The device may be of composite construction, utilizing polymer coating or applied sections that will enhance bone growth affecting a greater probability of fusion. Coatings may be selected that allow incorporation of bone growth stimulus factors. An additional property of composite construction may be the capability preventing the formation of wear debris or providing for the sequestration of wear debris from the immune system of the patient. This may be achieved by entirely coating each shape memory component 122 with a material is impermeable to the immune system. FIG. 12. The material may be a polymer or polymers. Alternately, the device may be sequestered within a bag, balloon, or other sealable containment that forms a liner 132 for the surgically created osteotomy 134 and contains the deployed device 136. FIG. 13.

What is claimed is:

1. A thermally active therapeutic device for implantation into a vertebra, comprising: a thermally active member of indeterminate shape below a transition temperature, and said thermally active member having a determinate, coiled shape above said transition temperature, wherein said determinate, coiled shape occupies a space within a vertebral body, and wherein said transition temperature is a predetermined temperature.

2. A thermally active therapeutic device for implantation into a vertebra as described in claim 1, wherein said thermally active member comprises plural determinate coiled elements when said thermally active member is above said transition temperature.

3. A thermally active therapeutic device for implantation into a vertebra as described in claim 1, wherein an external profile of said thermally active member is shaped to occupy substantially all of a void between two vertebral bodies.

4. A thermally active therapeutic device for implantation into a vertebra as described in claim 2, wherein an external profile of said thermally active member is shaped to occupy substantially all of a void between two vertebral bodies.

5. A thermally active therapeutic device for implantation into a vertebra as described in claim 1, wherein said thermally active member is encapsulated.

6. A thermally active therapeutic device for implantation into a vertebra as described in claim 1, wherein said thermally active member is encapsulated in a polymer.

7. A thermally active therapeutic device for implantation into a vertebra as described in claim 1, wherein said thermally active member comprises a biologic fusion enhancement agent.

8. A thermally active therapeutic device for implantation into a vertebra as described in claim 1, wherein said thermally active member is present within a container when implanted in said vertebra.

9. A thermally active therapeutic device for implantation into a vertebra as described in claim 1, wherein said thermally active member is present within a balloon when implanted in said vertebra.

10. A thermally active therapeutic device for implantation into a vertebra as described in claim 1, wherein said thermally active member comprises a textured outer surface.

11. A thermally active therapeutic device for implantation into a vertebra as described in claim 1, wherein said thermally active member comprises an attenuated side.

12. A thermally active therapeutic device for implantation into a vertebra as described in claim 1, wherein said thermally active member comprises an internal coiled structure, and an external coiled structure that surrounds said internal coiled structure.

13. A thermally active therapeutic device for implantation into a vertebra as described in claim 1, wherein said thermally active member that is below said transition temperature is capable of being translocated through an elongated lumen.

14. A thermally active therapeutic device for implantation into a vertebra as described in claim 1, wherein said thermally active member is formed as a cage when said thermally active member is above said transition temperature.

15. A thermally active therapeutic device for implantation into a vertebra as described in claim 1, wherein said thermally active member is capable of manual formation into an elongated substantially linear shape when said thermally active member is below said transition temperature.

16. A thermally active therapeutic device for implantation into a vertebra as described in claim 1, wherein said thermally active member is capable of manual formation into an elongated substantially linear shape when said thermally active member is below said transition temperature and said thermally active member is formed as a cage when said thermally active member is above said transition temperature.

17. A thermally active therapeutic device for implantation into a vertebra as described in claim 1, wherein said thermally active member that is below said transition temperature is capable of being translocated through an elongated lumen that is present within a flexible tube.

18. A thermally active therapeutic device for implantation into a vertebra as described in claim 1, wherein a void between two vertebral bodies into which said thermally active member is positioned comprises a space occupying and bone growth stimulating material.

19. A thermally active therapeutic device for implantation into a vertebra as described in claim 1, wherein said thermally active member that is below said transition temperature is capable of being formed in a substantially linear shape and translocated through an elongated lumen.

20. A thermally active therapeutic device for implantation into a vertebra as described in claim 1, wherein said thermally active member applies a force to a vertebral body to modify an anatomic relationship of said vertebral body.

21. A thermally active therapeutic device for implantation into a vertebra as described in claim 1, wherein said predetermined temperature is a temperature below a body temperature of a vertebrate into which the thermally active member is implanted.

22. A thermally active therapeutic device for implantation into a vertebra as described in claim 1, wherein said thermally active member is positioned between two vertebral bodies and said thermally active member changes in shape in response to a shift of a dynamic load axis between two vertebral bodies.

23. A thermally active therapeutic device for implantation into a vertebra as described in claim 1, wherein said thermally active member is positioned between two vertebral bodies, and said thermally active member changes in shape in response to rotation of one of said two vertebral bodies relative to a second of said two vertebral bodies, and said thermally active member permits rotation of one of said two vertebral bodies relative to a second of said two vertebral bodies.

24. A thermally active therapeutic device for implantation into a vertebra as described in claim 1, wherein said thermally active member is positioned between two vertebral bodies, and said thermally active member changes in shape in response to lateral movement of one of said two vertebral bodies relative to a second of said two vertebral bodies, and said thermally active member permits lateral movement of one of said two vertebral bodies relative to a second of said two vertebral bodies.

25. A thermally active therapeutic device for implantation into a vertebra as described in claim 1, wherein said thermally active member is positioned between two vertebral bodies, and said thermally active member changes in shape in response to anterior and posterior movement of one of said two vertebral bodies relative to a second of said two vertebral bodies, and said thermally active member permits anterior and posterior movement of one of said two vertebral bodies relative to a second of said two vertebral bodies.

26. A thermally active therapeutic device for implantation into a vertebra as described in claim 1, wherein said thermally active member is positioned between two vertebral bodies, and said thermally active member changes in shape in response to tensile movement of one of said two vertebral bodies relative to a second of said two vertebral bodies, and said thermally active member permits tensile movement of one of said two vertebral bodies relative to a second of said two vertebral bodies.

27. A thermally active therapeutic device for implantation into a vertebra as described in claim 1, wherein said thermally active member is positioned between two vertebral bodies and an annular ligament.

28. A thermally active therapeutic device for implantation into a vertebra as described in claim 1, wherein said thermally active member is positioned between two vertebral bodies and an annular ligament, wherein physiological function of said annular ligament is materially preserved after said thermally active member is positioned.
